(19) United States
(12) Patent Application Publication
(10) Pub. No.: US 2022/0249419 A1
(43) Pub. Date: Aug. 11, 2022

(54) C5 KETONE COMPOSITIONS, AND RELATED METHODS, FOR THERAPEUTIC AND PERFORMANCE SUPPLEMENTATION

(71) Applicant: Keto Innovations, LLC, Melissa, TX (US)

(72) Inventors: Ryan P. Lowery, Tampa, FL (US); Jacob Wilson, Tampa, FL (US); Shawn Wells, Frisco, TX (US); Kylin Liao, Frisco, TX (US)

(73) Assignee: Keto Innovations, LLC, Melissa, TX (US)

(21) Appl. No.: 17/732,482

(22) Filed: Apr. 28, 2022

Related U.S. Application Data
(63) Continuation-in-part of application No. 16/306,412, filed on Nov. 30, 2018, now Pat. No. 11,337,945.
(60) Provisional application No. 62/343,941, filed on Jun. 1, 2016.

(51) Int. Cl. A61K 31/20; A61K 31/19

(52) U.S. Cl. CPC A61K 31/20; A61P 43/00; A61K 31/19

(57) ABSTRACT

The present disclosure pertains to compositions and methods for the treatment and/or prevention of one or more of obesity, diabetes, metabolic syndrome, Alzheimer's disease, Chronic Fatigue Syndrome (CFS), aging, fibromyalgia, dyslipidemia, hypercholesterolemia, dyslipidemia, Parkinson's disease, migraines, Traumatic Brain Injury (TBI), Attention Deficit Disorder (ADD)/Attention Deficit Hyperactivity Disorder (ADHD), Cancer, Cardiovascular Disease (CVD)/Coronary Artery Disease (CAD), Chronic Pain, neuralgia, depression, amyotrophic lateral sclerosis (ALS), and epilepsy, Insufficient Cellular Energy (ICE) and mitochondrial dysfunction. The present disclosure also pertains to methods for increasing mental and/or physical performance levels and/or decreasing exertion during exercise in a subject by the administration of C5 ketones.

### CROSS-REFERENCE TO RELATED APPLICATIONS

This application is a continuation in part of U.S. patent application Ser. No. 16/306,412 which was filed on Nov. 30, 2018 and is entitled “C5 KETONE COMPOSITIONS, AND RELATED METHODS, FOR THERAPEUTIC AND PERFORMANCE SUPPLEMENTATION”, which is a U.S. National Stage of International Application No. PCT/M2017/054679, filed Aug. 1, 2017, which designates the U.S., published in English, and claims the benefit of U.S. Provisional Application Ser. No. 62/343,941, filed Jun. 1, 2016, all of which are hereby incorporated by reference for all purposes.

### BACKGROUND

Beta-hydroxybutyrate (BHB) (a C4 ketone) and its salts, esters, and combinations thereof have become increasingly common in the marketplace for the ability to increase blood BHB levels, promote increases in cognitive function, and an array of benefits that may benefit diseased individuals.

The heart, brain and kidney are the three organs with the highest activity of enzymes utilizing ketone bodies (Ardawi and Newsholme 1982). For instance, studies have shown an improvement in the neurological status of a newborn with pyruvate carboxylase deficiency during treatment with triheptanoin (C7 ketone) (Mochel et al 2005; Roe and Mochel 2006).

### SUMMARY

In various implementations, a therapeutically effective amount of one or more C5 ketone body, pharmaceutically acceptable salt, ester, chelate thereof, and/or combinations thereof may be administered to increase mental and/or physical performance levels in a subject. Administration may increase and/or maintain ketosis, increase blood ketone levels, increase and/or maintain weight loss, increase fat loss, improve body composition, increase strength (e.g., power, muscle mass), increase energy, and/or increase cognitive health (e.g., cognitive function, memory, focus, motor function associated with age or disease related declines, etc), glucose tolerance, maintain and/or reduce blood glucose levels, reduce or inhibit progression of symptoms of traumatic brain injury, and/or reduce or inhibit progression of cognitive decline symptoms. The pharmaceutically effective amount of C-5 ketone bodies may be administered to healthy and/or not healthy (e.g., with diseases such as obesity, etc. and/or disorders) individuals.

In some implementations, the methods described aid Kreb cycle function and/or anaplerosis in a subject being treated with the described compositions. In some implementations, the C5 ketone body is β-ketopentanoate (beta-ketopentanoate).

In some implementations, the C5 ketone body may be administered with one or more additional compounds (beta-hydroxybutyrates, fatty acids, triglycerides, vitamins, minerals, amino acids, proteins, etc.). The C5 ketone body may be administered with one or more pharmaceutically acceptable binders and/or carriers.

The compositions that include the C5 ketone body may be administered to treat of the diseases, conditions, and/or symptoms thereof as described herein. In various implementations, approximately 1 to approximately 50 g of the C5 ketone body, pharmaceutically acceptable salt, ester, chelate thereof, and/or combinations thereof may be administered (e.g., orally) to an individual.

The details of one or more implementations are set forth in the accompanying drawings and the description below. Other features, objects, and advantages of the implementations will be apparent from the description and drawings.

### BRIEF DESCRIPTION OF THE DRAWINGS

For a more complete understanding of this disclosure and its features, reference is now made to the following description, taken in conjunction with the accompanying drawings, in which:

FIG. 1 illustrates the metabolism of C5 ketones in their racemic mixture and dissociated into their respective isoforms.

FIG. 2 illustrates C4 ketogenesis and C5 ketogenesis in the liver. Numbers refer to the following enzymes: 3-ketoacyl-CoA thiolase (1); HMG-CoA synthase (2); HMG-CoA lyase (3); and beta-hydroxybutyrate dehydrogenase(4). The figure also shows the link between propionyl-CoA and the CAC via anaplerosis.

FIG. 3 illustrates the metabolism of triheptanoin into C5 ketone bodies 3-hydroxypentanoate (BHP) and beta-ketopentanoate (BKP), which ultimately get transported out of the mitochondria.

FIG. 4 shows a graph of blood glucose level with BKP administration.

FIG. 5 shows a graph of blood ketone level with BHP administration.

FIG. 6 shows a graph of visual analog scales (VAS) of well-being with BKP administration.

FIG. 7 shows a graph of VAS of energy with BKP administration.

FIG. 8 shows a graph of VAS of focus with BKP administration.

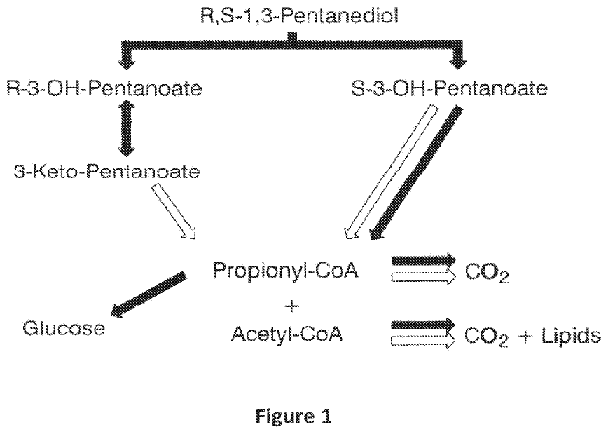

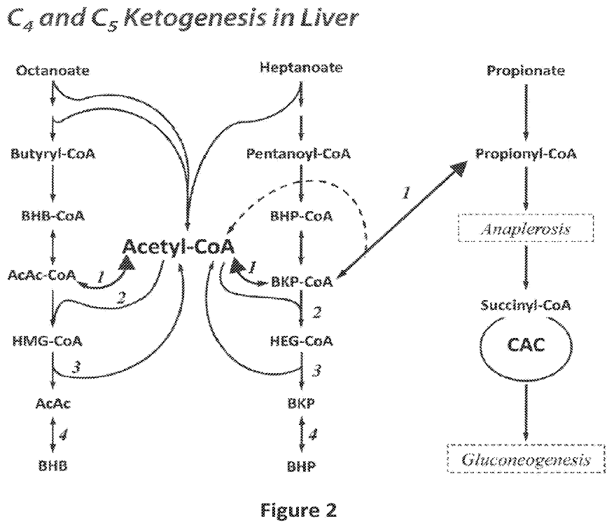

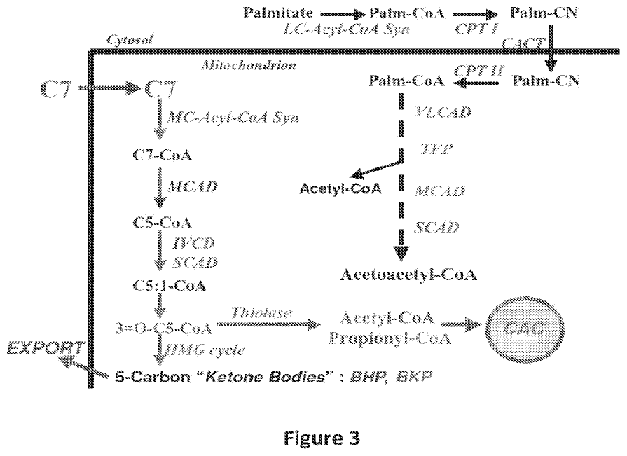

Like reference symbols in the various drawings indicate like elements.

### DETAILED DESCRIPTION

In various implementations, a C5 composition that includes one or more C5 ketone bodies may be administered to a subject in a therapeutically effective amount. The C5 composition may be orally administered. The C5 composition may include one or more additional compounds.

In various implementations, a therapeutically effective amount of one or more C5 ketone body, pharmaceutically acceptable salt, ester, chelate thereof, and/or combinations thereof may be administered to increase mental and/or physical performance levels in a subject. Administration may increase and/or maintain ketosis, increase blood ketone levels, increase and/or maintain weight loss, increase fat loss, improve body composition, increase strength (e.g., power, muscle mass), increase energy, and/or increase cognitive health (e.g., cognitive function, memory, focus, motor function associated with age or disease related declines, etc), glucose tolerance, maintain and/or reduce blood glucose levels, reduce or inhibit progression of symptoms of traumatic brain injury, and/or reduce or inhibit progression of cognitive decline symptoms. The pharmaceutically effective amount of C-5 ketone bodies may be administered to healthy and/or not healthy (e.g., with diseases such as obesity, etc. and/or disorders) individuals. In various implementations, compositions and methods may be utilized for the treatment and/or prevention of one or more of obesity; diabetes; metabolic syndrome; Alzheimer's disease; Chronic Fatigue Syndrome (CFS); aging; fibromyalgia; dyslipidemia; hypercholesterolemia; dyslipidemia; Parkinson's disease; migraines; Traumatic Brain Injury (TBI); Attention Deficit Disorder (ADD)/Attention Deficit Hyperactivity Disorder (ADHD); Cancer, such as for example, glioblastoma; Cardiovascular Disease (CVD)/Coronary Artery Disease (CAD); Chronic Pain; neuralgia; depression; amyotrophic lateral sclerosis (ALS); epilepsy; Insufficient Cellular Energy (ICE); mitochondrial dysfunction; and/or combinations thereof. Various methods may increasing mental and/or physical performance levels.

Any appropriate C5 ketone body may be utilized. For example, C5 ketone body may include 3-hydroxypentanoate (BHP) and/or beta-ketopentanoate (BKP). C5 ketones may include C5 ketone bodies that are unbound in their “free acid” state. C5 ketones may include C5 ketone salts (e.g. sodium, calcium, magnesium, etc.), esters (e.g. glycerol), chelates (e.g. amino acids), or any other appropriate chemical variant with C5 ketones attached.

In some implementations, C5 ketones can be in combination with other compounds, including other carbon length ketones themselves.

In some implementations, the C5 ketone may include C5 ketones such as beta-hydroxypentanoate (BHP) [R,S, & RS isoforms] and/or 3-ketopentanoate (BKP) (also known as 3-hydroxyvalerate and 3-ketovalerate). In some implementations, the C5 ketone bodies may include, but are not limited to, β-hydroxypentanoate (BHP) or a ester thereof; β-ketopentanoate (BKP) or a ester thereof β-hydroxypentanoate or a chelate thereof; β-ketopentanoate or a chelate thereof. In some implementations one or more of the C5 ketone bodies may include ester derivatives including alkyl ester derivatives, such as methyl, ethyl, propyl, butyl, hexyl, etc. Ester derivatives may include as one or more esters, such as mono-, di-, tri-, oligo-, and/or polyesters. For example, the C5 ketone bodies may include esters such as mono-esters of ethanol, mono-ester of 1-propanol, mono-ester of 1,2-propanediol, di-ester of 1,2-propanediol, mono-ester of 1,3-propanediol, di-ester of 1,3-propanediol, mono-ester of S—, R-, or S—R-1,3-butanediol, di-ester of S—, R-, or S—R-1,3-butanediol, mono-ester of glycerin, mono-ester, di-ester of glycerin, tri-ester of glycerin, and/or combinations thereof. C5 ketone bodies may be naturally formed in the liver from the partial oxidation of odd chain fatty acids (e.g. C-7, heaptanoic acid, like macadamia nut oil). C5 ketones have been shown to occur in trace amounts in human plasma and most plants. They are derived from the oxidation in liver of the R-moiety of R,S-1,3-pentanediol, a potential nutrient. Whether administered by bolus or constant infusion, the two substrates are interconverted and rapidly metabolized in an animal model.

Researchers found at least during short-term experiments, 3-hydroxypentanoate (BHP) and beta-ketopentanoate (BKP) are well metabolized in animals without apparent intolerance to a large supply of propionyl-CoA. In nondiabetic animals, C5 ketone bodies ((R)-BETA-hydroxypentanoate and beta-ketopentanoate) are very rapidly used when administered intravenously at doses corresponding to 75% of the energy requirement (Leclerc et al 1995).

In some implementations, at least a portion of the C5 ketone bodies may be interconverted by mitochondrial BHB dehydrogenase. In peripheral tissues, C5 ketone bodies may be at least partially converted to propionyl-CoA (which is anaplerotic). Peripheral tissues may have a high capacity to utilize exogenous C5 ketone bodies, especially heart, kidney, and brain, which have high activities of 3-oxoacid-CoA transferase. C5 ketone bodies may provide a unique benefit in promoting increases in mental and physical performance, aiding Kreb cycle function, and contributing as a unique aide in various diseased populations (e.g., obesity, diabetes, cancer, cardiovascular disease, epilepsy, Alzheimer's, Parkinson's, etc.) and anaplerotic scenarios. C5 ketone bodies may improve weight loss/fat loss.

In various implementations the C5 composition may include approximately 0.5 g to approximately 50 g of one or more C5 ketone bodies. The C5 ketone bodies of the C5 composition may include one or more C5 ketone bodies such as BHP and/or BKP.

In various implementations, a C5 ketone body may include salts, esters, chelates, complexes, other appropriate forms, and/or combinations thereof of one or more C5 ketone bodies. For example, a C5 ketone body may include salts, esters, chelates, complexes, other appropriate forms, and/or combinations thereof of BHP. A C5 ketone body may include salts, esters, chelates, complexes, other appropriate forms, and/or combinations thereof of BKP. A C5 ketone body may include free C5 ketone bodies such as free BHP and/or free BKP. In some implementations, the C5 ketone bodies may include racemic mixtures and/or enriched mixtures (e.g., one form may be predominant) to produce a predetermined effect during administration. For example, the C5 ketones utilized in the methods and compositions may be provided in an isolated or purified form. Synthetic variants of the C5 ketones may be utilized, either individually or in combination with other synthetic C5 ketone variants, or in combination with non-synthetic variants.

In some implementations, the C5 composition may include C5 ketone body salts. The cation associated with the C5 ketone body salts may be any appropriate cation such as sodium, potassium, magnesium, calcium, zinc, selenium, chromium, other appropriate minerals, and/or combinations thereof. The cations may be chosen to produce a specific effect in the body. For example, one or more of the cations in the C5 composition may operate in a similar manner as an electrolyte drink (e.g., by utilizing a predetermined amount of sodium, potassium, calcium, and/or magnesium). In some implementations, two or more, three or more, and/or four or more different C5 ketone body salts may be selected to produce a predetermined effect in the body (e.g., high initial spike in blood ketone levels, approximately even increase in blood ketone levels for a predetermined period of time such as 60 and/or 90 minutes, etc., a combination with low magnesium to reduce GI issues, a combination with relatively higher magnesium to facilitate sleep while maintaining C5 composition administration schedule(s), etc.).

In some implementations, the C5 composition may include isomers and/or polymers of C5 ketone bodies, as appropriate.

In various implementations, a pharmaceutically effective amount of the C5 composition including one or more C5 ketone bodies may be administered in an individual to promote and/or maintain ketosis, cause weight loss and/or manage weight, and/or increase blood ketone levels. For example, approximately 0.1 g to approximately 50 g of BHP and/or BKP may be administered to a subject. In some implementations, approximately 0.1 g to approximately 15 g of BHP and/or BKP may be administered to an individual. In some implementations, approximately 1 g to approximately 10 g of BHP and/or BKP may be administered, for example, once a day to 5 times a day. In some implementations, less than 3 g of C5 ketone bodies may be administered to the subject (e.g., approximately 0.1 to approximately 3 g of BHP and/or BKP). The administration may cause weight loss and/or maintenance; elevated beta-hydroxybutyrate levels in the blood; elevated, reduced, and/or maintenance of blood ketone levels; induction and/or maintenance of ketosis; and/or reduction; improve mental acuity; improve focus; improve energy; improve cognitive function; reduce traumatic brain injury; improve diabetes; improve glucose tolerance; decrease blood glucose levels; reduce neurological disorders and/or symptoms thereof; improve cancer and/or symptoms thereof; improve inflammatory conditions; suppressing appetite; improve symptoms associated with aging; provide anti-glycation affects; improve epilepsy and/or symptoms thereof; improve depression and/or symptoms thereof; improve performance; improve strength; increase muscle mass; increase fat loss; improve body composition; improve energy; improve focus; improve cognitive function; improve mood and/or well-being; and/or combinations thereof. The C5 composition that includes one or more C5 ketone bodies may be administered in healthy and not healthy individuals (e.g., individuals with diseases and/or disorders).

In some implementations, blood ketone levels and/or blood glucose levels may be reduced and/or maintained within a predetermined range when a pharmaceutically effective amount of the C5 composition is administered. In some implementations, a health of an individual (e.g., strength, symptoms of disease, mental acuity, fasting glucose levels, etc.) may be improved and/or maintained by administration of the C5 composition.

In some implementations, the C5 compositions comprising one or more C5-ketone body, or pharmaceutically acceptable salt, ester, or chelate thereof, as the active ingredient. The compositions described can also be in the form of a pharmaceutical composition comprising one or more pharmaceutically acceptable carriers described herein. The compounds and compositions of the disclosure can be used to treat, alleviate, inhibit or prevent the diseases and conditions further described herein. Further, the compounds and compositions of the disclosure can be used for increasing mental and/or physical performance levels in a subject or for aiding in weight loss. In some implementations, the C5 ketone body, or pharmaceutically acceptable salt, ester, or chelate thereof, concentration is from about 1 gram to about 50 grams. In some implementations, the C5 ketone, or pharmaceutically acceptable salt, ester, or chelate thereof, concentration is from about 1 gram to about 40 grams. In some implementations, the C5 ketone, or pharmaceutically acceptable salt, ester, or chelate thereof, concentration is from about 1 gram to about 30 grams. In some implementations, the C5 ketone, or pharmaceutically acceptable salt, ester, or chelate thereof, concentration is from about 1 gram to about 20 grams. In some implementations, the C5 ketone, or pharmaceutically acceptable salt, ester, or chelate thereof, concentration is from about 1 gram to about 10 grams. In some implementations, the C5 ketone, or pharmaceutically acceptable salt, ester, or chelate thereof, concentration is from about 10 grams to about 50 grams. In some implementations, the C5 ketone, or pharmaceutically acceptable salt, ester, or chelate thereof, concentration is from about 20 grams to about 50 grams. In some implementations, the C5 ketone, or pharmaceutically acceptable salt, ester, or chelate thereof, concentration is from about 30 grams to about 50 grams. In some implementations, the C5 ketone, or pharmaceutically acceptable salt, ester, or chelate thereof, concentration is from about 40 grams to about 50 grams. In some implementations, the C5 ketone, or pharmaceutically acceptable salt, ester, or chelate thereof, concentration is about 10 grams.

In some implementations, the C5 composition may include one or more additional compounds.

In some implementations, the C5 composition may include and/or may be administered with, and/or coupled to a compound such as an amino acid. For example, the C5 composition may include one or more amino acids such as leucine, taurine, purine, lysine, arginine, histidine, ornithine, creatine, agmatine, citrulline and/or combinations thereof. In some implementations, Administration of C5 ketone bodies with amino acids may reduce the intake of cations associated with salts (e.g., which may inhibit side effects associated with administration) and/or allow administration of another compound that has health benefits (e.g., administration of some amino acid may promote smooth muscle growth and/or cell repair). In some implementations, approximately 0.5 g to approximately 10 g of amino acid may be administered with one or more C5 ketone bodies. For example, less than approximately 50 g of C5 ketone body may be administered with less than approximately 60 mg of an amino acid, such as leucine, may be administered daily. In some implementations, approximately 0.5 g to approximately 2 g of an amino acid, such as leucine, may be administered with a C5 ketone body. For example, approximately the composition administered may include approximately 0.1 to approximately 7 g C5 ketone body and approximately 1-3 g of leucine. The C5 ketone body and the leucine may be a mixture; administered separately and proximate in timing; a complex, and/or administered in any other appropriate manner.

In some implementations, the C5 composition may include C5 ketone body salt and one other slower blood ketone elevating C5 ketone body. For example, an individual may be administered a first weight amount of BHP salt and/or BKP salt and a second weight amount of ester of C5 ketone body and/or a polymer of C5 ketone body. The first amount and the second amount may be different or the same.

In some implementations, the C5 composition may include one or more beta-hydroxybutyrates. In some implementations, R-beta-hydroxybutyrate may be administered to manage glucose tolerance, reducing fasting glucose levels, and/or maintain and/or promote ketosis (e.g., maintain and/or increase blood ketone levels). As described in herein and in U.S. patent application Ser. Nos. 15/491,924 and 17/367,206, to Lowery et al. and entitled “Administration of Beta-hydroxybutyrate and Related Compounds in Humans”, which is incorporated by reference to the extent that the teachings do not conflict with the present disclosure. Also as described in herein and in U.S. patent application Ser. No. ______ filed Apr. 22, 2022 to Lowery et al. and entitled “ADMINISTRATION OF R-BETA-HYDROXYBUTYRATE SALT BLEND AND RELATED COMPOUNDS IN HUMANS”, which is incorporated by reference to the extent that the teachings do not conflict with the present disclosure.

In some implementations, C5 composition may include one or more additional compounds that may or may not be capable of independently increasing ketone levels, maintaining ketone levels, inducing ketosis, and/or maintaining ketosis. For example, additional compounds capable of independently increasing blood ketone levels may include short chain fatty acids (e.g., fatty acid with between 2 carbons than 6 carbons), short chain triglycerides (e.g., triglycerides with less than 6 carbons), medium chain fatty acids (e.g., fatty acid with 6-12 carbons), medium chain triglycerides (e.g., triglycerides with 7-12 carbons), long chain fatty acids (e.g., fatty acids with more than 12 carbons), long chain triglycerides (e.g., triglycerides with more than 12 carbons), and/or combinations thereof. In some implementations, short chain fatty acids and/or triglycerides may include acetate, propionate, and/or butyrate. Medium chain fatty acids and/or triglycerides may include lauric acid and/or coconut oil, coconut milk powder, fractionated coconut oil, isolated hexanoic acid, isolated octanoic acid, isolated decanoic acid, ethoxylated triglyceride, triglyceride derivatives thereof, aldehyde triglyceride derivatives thereof, monoglyceride derivatives thereof, diglyceride derivatives thereof, triglyceride derivatives thereof, and/or alkyl esters thereof. Long chain fatty acids and/or triglycerides may include dairy products and/or palm oil. In some implementations, a composition including C5 ketone bodies and an additional compound that is independently capable of increasing ketone levels may increase ketone levels greater than merely the capability of each component individually (e.g., greater than an additive increase). For example, the C5 composition may include C-5 ketone bodies and/or and an additional compound independently capable of increasing ketone levels such as caffeine. The C5 composition may include C5 ketone bodies, R-beta-hydroxybutyrate, and/or and an additional compound independently capable of increasing ketone levels such as caffeine. In some implementations, the composition may include approximately 0.5 mg to approximately 50 g of C5 ketone bodies (e.g., BHP salt and/or BKP salt) and caffeine. In some implementations, the C5 composition may include approximately 0.5 mg to approximately 15 g of C5 ketone bodies and less than approximately 500 mg of caffeine. In some implementations, the C5 composition may include approximately 0.5 mg to approximately 15 g of C5 ketone bodies and approximately 5 mg to approximately 500 mg of caffeine. In some implementations, the composition may include approximately 0.5 mg to approximately 15 g of C5 ketone bodies and approximately 10 mg to approximately 150 mg of caffeine. In some implementations, the composition may include approximately 0.5 mg to approximately 15 g of C5 ketone bodies and approximately 10 mg to approximately 50 mg of caffeine. The C5 composition with C5 ketone bodies (e.g., BHP salt and/or BKP salt, esters of BHP and/or BKP) and caffeine may increase and or maintain ketosis, weight loss, fat loss, and/or mental acuity. In some implementations, the C5 composition with caffeine may increase mental processes (e.g., acuity including cognitive functioning, mood, energy, alertness, focus, performance, effects of aging, etc.); improve and/or maintain body composition; function as a therapeutic for one or more of the described conditions or disorders (e.g., treat neurological disorders); and/or combinations thereof. In some implementations, the C5 composition may include an additional compound independently capable of increasing ketone levels, such as 1,3,7,9-Tetramethyluric acid (commercially available as theacrine; and/or commerically available as TeaCrine® from Compound Solutions, California, USA). In some implementations, the composition may include approximately 0.5 mg to approximately 15 g of C5 ketone bodies and less than approximately 500 mg of 1,3,7,9-Tetramethyluric acid. In some implementations, the C5 composition may include approximately 5 mg to approximately 15 g of C5 ketone bodies and less than approximately 500 mg of 1,3,7,9-Tetramethyluric acid.

In some implementations, the C5 composition may include approximately 0.5 to approximately 50 g of C5 ketone bodies and 0.5 to 20 g of beta-hydroxybutyrate (e.g., free BHB, free RBHB, sodium beta-hydroxybutyrate, potassium beta-hydroxybutyrate, calcium beta-hydroxybutyrate, etc.). The combination of the C5 ketone bodies and the beta-hydroxybutyrate may provide unexpected increase in blood ketone levels when compared to the components individually and/or at least partially enter the Krebs cycle.

In some implementations, the C5 composition may include one or more fatty acids, esters, or triglycerides. For example, the C5 composition may include a short chain compound (e.g., short chain fatty acid and/or short chain triglyceride) may unexpectedly increase ketone concentrations in the blood more than the administration of similar amounts of C5 ketone bodies and medium chain compounds (e.g., short chain fatty acid and/or short chain triglyceride) and/or may increase ketone concentrations in the blood more than each component individually.

In some implementations, the C5 composition may include odd chain fatty acids. The administration of C5 compositions with odd chain fatty acids (e.g., C7 fatty acid) may increase C5 production in the liver.

In some implementations, a pharmaceutically effective amount of C5 composition may include a pharmaceutically effective amount of long chain fatty acid and/or triglyceride. For example, 0.1-50 g of C5 ketone bodies and 0.1 to 50 g of long chain fatty acid may be administered to an individual between 1-5 times a day. In some implementations, approximately 1 g to approximately 3 g of C5 ketone bodies and approximately 1 g of long chain fatty acid and/or triglyceride may be administered from once a day to approximately 5 times a day.

In some implementations, C5 ketone bodies, short chain compound(s) (e.g., fatty acids and/or triglycerides, butyrate), and/or medium chain compound(s) (e.g., fatty acids and/or triglycerides) may be administered approximately simultaneously and/or sequentially to an individual. For example, approximately 0.1 g to approximately 50 g C5 ketone bodies, approximately 0.1 g to approximately 50 g short chain triglyceride, and approximately 0.1 g to approximately 50 g medium chain fatty acid such as lauric acid and/or coconut oil may be administered between 1-5 times a day. In some implementations, approximately 1 g to approximately 3 g of C5 ketone bodies and approximately 1 g of short chain fatty acid and/or triglyceride and/or approximately 1 g of medium chain fatty acid and/or triglyceride may be administered from once a day to approximately 5 times a day. In some implementations, approximately 0.1 g to approximately 20 g C5 ketone bodies (e.g., salts, esters, isomers, and/or other appropriate forms) may be administered in humans.

In some implementations, other compounds, such as compounds capable of independently decreasing glucose levels, may be administered with beta-hydroxybutyrate, such as berberine and/or associated metabolites (e.g., dihydroberberine and/or tetrahydroberberine). U.S. patent application Ser. No. 15/491,933 entitled “ADMINISTRATION OF DIHYDROBERBERINE” to Lowery et al, filed Apr. 19, 2017 and U.S. Provisional Patent Application No. 62/324,794, entitled “ADMINISTRATION OF DIHYDROBERBERINE” to Lowery et al, filed Apr. 19, 2016, describe dihydroberberine administration with ketone sensitizers such as beta-hydroxybutyrate, and is hereby fully incorporated herein. U.S. patent application Ser. No. 17/341,312 entitled “ADMINISTRATION OF BERBERINE METABOLITES” to Lowery et al, filed Jun. 7, 2021, describe dihydroberberine administration with ketone sensitizers such as beta-hydroxybutyrate, and is hereby fully incorporated herein. In some implementations, one or more C5 ketone bodies, beta-hydroxybutyrates, and/or other compounds described herein may be utilized as a ketone sensitizer with the dihydroberberine.

In some implementations, the C5 ketone bodies may include or be administered with one or more other compounds such as, but not limited to, amino acids, amino acid metabolites, proteins (e.g., dairy proteins such as whey and/or casein; vegan proteins such as pea protein and/or pumpkin protein; egg protein; meat derived proteins; any other appropriate protein; and/or combinations thereof), vitamins, minerals, herbs and/or extracts of herbs, coconut milk (e.g., powder), flavorings, colorings, binders, electrolytes, tetrahydrobiopeterin, nucleic acids, alpha-ketoglutaric acid, alpha lipoic acid, nutritional co-factors, beta-methyl-beta-hydroxybutyrate, arginine alpha-ketoglutarate, R-alpha lipoic acid, thiamine, NAD+, NADH, riboflavin, FAD+, FADH, riboflavin-5-phosphate, niacin, nicotinic acid, niacinamide, inositol hexanicotinate, pyridoxine, pyridoxal, pyridoxamine, ascorbic acid and ascorbate salts, citric acid, malic acid, sodium benzoate, Pyridoxal-5-Phosphate, methylcobalamin, cyanocobalamin, adenosylcobalamin, hydroxycobalamin, pantothenic acid, pantetheine, potassium sorbate, acesulfame K, aspartame, sucralose, stevia, monk fruit extract, allulose, prebiotic fibers, XOS, GOS, MOS, IMO, LOS, xanthan gum and other organic gums/thickeners/suspension agents, and combinations thereof. In some implementations, the R-beta-hydroxybutyrate salt blend may include collagen peptides and/or hyaluronic acid.

In some implementations, the C5 composition may include and/or be administered in a bone broth (e.g., powdered and/or in fluid form). The inclusion of bone broth may increase satiety and/or palatability. The bone broth and C5 ketone bodies may increase and/or facilitate maintenance of ketosis. For example, C5 composition may include approximately 0.5 to approximately 50 g of C5 ketone bodies compounds and approximately 1 g to approximately 50 g of bone broth. As another non-limiting example, C5 composition may include approximately 0.5 to approximately 20 g of C5 ketone bodies and approximately 1 g to approximately 30 g of bone broth. As another non-limiting example, C5 composition may include approximately 0.5 to approximately C5 ketone bodies and approximately 1 g to approximately 10 g of bone broth. The C5 composition that includes bone broth may include additional compounds such as caffeine, vitamins, collagen peptides, etc. The C5 composition that includes bone broth may cause greater user satisfaction when compared with conventional bone broths (e.g., due to the pleasant saltiness provided by the C5 salts, ability to increase ketone delivery without increasing fat intake, the appetite suppressant of the C5 ketone bodies, etc.). Since bone broth may be provided as a low-fat bone broth, the c5 composition that includes bone broth may provide users that are unable to tolerate a high fat diet, benefits of the C5 composition and C5 ketone bodies, and satiety without a high fat intake. The C5 composition that includes bone broth may be provided in a packet (powdered and/or fluid) and water may be added to the contents of the packet prior to consumption by the individual. The inclusion of bone broth may increase satiety, maintain ketosis, and/or increase mood and thus may increase compliance with an administration schedule and user satisfaction.

In some implementations, administration of the C5 composition may improve and/or maintain health markers such as C-reactive protein and/or fasting glucose. Administration of the C5 composition may decrease inflammation (e.g., as shown by C-reactive protein levels). Administration of C5 composition may decrease fasting glucose. For example, administration of approximately 0.5 to 50 g of C5 ketone bodies may cause a reduction in and/or maintain a low fasting glucose. In some implementations, the C5 composition may include and/or be administered with one or more other of the described additional compounds to decrease glucose levels and/or sensitivity. For example, a berberine, such as dihydroberberine, may cause reduce and/or maintain low fasting glucose. Administration of the C5 composition with a berberine, such as dihydroberberine, may cause reduce and/or maintain low glucose levels. In some implementations, less than approximately 15 g of C5 ketone bodies may be administered with less than approximately 600 mg of dihydroberberine.

In some implementations, additional compounds may not be capable of independently increasing blood ketone levels and/or decreasing blood glucose levels (e.g., additives, flavorings, colorings, minerals, vitamins, binders, anti-caking agents, etc.).

The C5 composition may be administered in any appropriate delivery form (e.g., tablet; capsule; food products such as powdered products that can be mixed into food, mixed into beverages, and/or consumed directly; beverage product; etc.). The C5 composition may be administered according to any appropriate schedule (e.g., periodic dosages, dosages as user desires, etc.). The C5 composition administration schedule may inhibit administration that elevates blood ketone levels too high, decreases blood glucose levels too low, and/or causes an individual to consume a dosage that substantially elevates the risk of adverse and/or side effects, in some implementations.

In some implementations, the C5 composition may include a long-acting component and/or be long-acting. For example, since the body digests polymers and/or esters of C5, the delivery of C5 ketone bodies may be slower than a digestion of a C5 ketone body salts. In some implementations, the composition may include a C5 ketone body salt and a long-acting C5 ketone body form (e.g., polymer, ester, coated and/or processed form to provide slow release). In some implementations, a first dose(s) may include at least one non-long-acting form of C5 ketone body and a second dose(s) may include at least one long-acting form of C5 ketone body. The first dose(s) may be administered to cause a predetermined health impact and the second dose(s) may be administered to maintain the caused predetermined health impact. In some implementations, users may select the appropriate dose based on user preference and/or properties (e.g., a user on a ketogenic diet may chose the second dose since the user may already be in ketosis).

In various implementations, the C5 composition may include one or more amino acids (e.g., leucine), caffeine, flavors, vitamins, minerals, herb extracts (e.g., extract of Uncaria tomentosa), and/or pharmaceutically acceptable binders (e.g., fluid and/or solid binders). The C5 composition may include bone broth. In some implementations, the C5 composition may include fatty acids, esters, or triglycerides (e.g., short chain, long chain, and/or medium chain). In some implementations, the C5 composition may include 0.5 to 3 g of C5 ketone bodies, caffeine, and/or fatty acids, esters, and/or triglycerides (e.g., medium, short, and/or long chain).

In some implementations, the C5 composition may include one or more NAD, ketone esters, Sweeteners (e.g., natural and/or synthetic), Flavors, Vitamins, CMED 100, AcAc, Caffeine, MCTs, SCFA, Creatine, Protein, Glucose, Glutamine, Electrolytes (e.g., additionally and/or from the salt of the C5 ketone bodies and/or beta-hydroxybutyrate), Pyruvate, Lactate, and/or combinations thereof.

In some implementations, the C5 composition may include cannabidiol (CBD) with or without R-beta-hydroxybutyrate. As described in U.S. patent application Ser. Nos. 16/667,851 and 17/771,561 entitled “Administration of Butyrate, Beta-hydroxybutyrate, cannabidiol, and related compounds in humans”, which is incorporated by reference for all purposes to the extent the teachings do not conflict with the teachings herein. For example, the C5 composition may include approximately 0.5 to approximately 50 g of C5 ketone bodies and approximately 5 mg to approximately 300 mg CBD. As another non-limiting example, the C5 composition may include approximately 0.5 to approximately 50 g of C5 ketone bodies, approximately 0.5 to approximately 20 g of beta-hydroxybutyrate, and approximately 5 mg to approximately 300 mg CBD.

In some implementations, methods for the treatment and/or prevention of one or more of obesity, diabetes, metabolic syndrome, Alzheimer's disease, Chronic Fatigue Syndrome (CFS), aging, fibromyalgia, dyslipidemia, hypercholesterolemia, dyslipidemia, Parkinson's disease, migraines, Traumatic Brain Injury (TBI), Attention Deficit Disorder (ADD)/Attention Deficit Hyperactivity Disorder (ADHD), Cancer, Cardiovascular Disease (CVD)/Coronary Artery Disease (CAD), Chronic Pain, neuralgia, depression, amyotrophic lateral sclerosis (ALS), and epilepsy, Insufficient Cellular Energy (ICE) and mitochondrial dysfunction, include administering a therapeutically effective amount of a C5 composition that includes one or more C5 ketone bodies, or pharmaceutically acceptable salt, ester, or chelate thereof to a subject (e.g., a mammal and/or a human). In some implementations administration of a pharmaceutically acceptable amount of the C5 composition may decrease the neurological decline and/or the symptoms of neurological decline related to age and/or disease (e.g., brain function, energy, focus, mood, motor control, etc.)

In some implementations administration of a pharmaceutically acceptable amount of the C5 composition may reduce inflammation.

C5 ketones have potential to work directly on the mitochondria, therefore, in some implementations, the C5 ketones and related methods described by this disclosure are useful for treating mitochondrial dysfunction. As such, C5 ketones are particularly useful in treating cancers that result from mitochondrial dysfunction, such as, for example, glioblastoma.

Aspects of the present disclosure may also be directed to methods for increasing blood ketone levels in a subject, comprising administering to the subject a therapeutically effective amount of a C5 ketone body, or pharmaceutically acceptable salt, ester, or chelate thereof, such as, for example, 3-hydroxypentanoate (BHP).

A further aspect of the present disclosure may be directed to methods for decreasing blood glucose levels in a subject, comprising administering to the subject a therapeutically effective amount of a C5 ketone body, or pharmaceutically acceptable salt, ester, or chelate thereof, such as, for example,3-ketopentanoate (BKP).

Aspects of the present disclosure are also directed to methods for increasing mental and/or physical performance levels which involve administering a therapeutically effective amount of a C5 ketone body, or pharmaceutically acceptable salt, ester, or chelate thereof, or pharmaceutical compositions of the present disclosure, to a subject, preferably a mammal, and most preferably a human. In some implementations, the C5 ketone body is (3-ketopentanoate. The mental performance level being increased may be a measure of well-being and/or focus. The physical performance level being increased may be a measure of energy.

In yet another aspect, the subject disclosure provides methods for aiding in weight loss in a subject, the methods comprising administering to the subject a therapeutically effective amount of a C5 ketone body, or pharmaceutically acceptable salt, ester, or chelate thereof

In a further aspect, the subject disclosure provides methods for lowering exertion during exercise in a subject, the methods comprising administering to the subject a therapeutically effective amount of a C5 ketone body (e.g., 3-ketopentanoate), or pharmaceutically acceptable salt, ester, or chelate thereof.

In some implementations, the exercise is moderate intensity exercise and in other implementations, the exercise is high intensity exercise. Moderate intensity exercise includes, for example, running, cycling, rowing, circuit training, and yoga. High intensity exercise includes, for example, sprinting, hockey, soccer, baseball, football, basketball, resistance training and boxing.

In some implementations, the C5 ketone, or pharmaceutically acceptable salt, ester, or chelate thereof, concentration utilized in the methods described is from about 1 gram to about 50 grams. In some implementations, the C5 ketone, or pharmaceutically acceptable salt, ester, or chelate thereof, concentration utilized in the methods described is from about 1 gram to about 40 grams. In some implementations, the C5 ketone, or pharmaceutically acceptable salt, ester, or chelate thereof, concentration utilized in the methods described is from about 1 gram to about 30 grams. In some implementations, the C5 ketone, or pharmaceutically acceptable salt, ester, or chelate thereof, concentration utilized in the methods described is from about 1 gram to about 20 grams. In some implementations, the C5 ketone, or pharmaceutically acceptable salt, ester, or chelate thereof, concentration utilized in the methods described is from about 1 gram to about 10 grams. In some implementations, the C5 ketone, or pharmaceutically acceptable salt, ester, or chelate thereof, concentration utilized in the methods described is from about 10 grams to about 50 grams. In some implementations, the C5 ketone, or pharmaceutically acceptable salt, ester, or chelate thereof, concentration utilized in the methods described is from about 20 grams to about 50 grams. In some implementations, the C5 ketone, or pharmaceutically acceptable salt, ester, or chelate thereof, concentration utilized in the methods described is from about 30 grams to about 50 grams. In some implementations, the C5 ketone, or pharmaceutically acceptable salt, ester, or chelate thereof, concentration utilized in the methods described is from about 40 grams to about 50 grams. In some implementations, the C5 ketone, or pharmaceutically acceptable salt, ester, or chelate thereof, concentration utilized in the methods described is about 10 grams.

Administration can be locally (confined to an area of body) and/or systemically in the subject. It may be desirable to administer the C5 ketones, or pharmaceutical compositions comprising same, locally to the area in need of treatment. This method of administration may be achieved by, for example, and not by way of limitation, local infusion, topical application, by injection, by catheter, or by means of an implant (e.g., a porous membrane). Administration may also be orally, such as but not limited to, by way of pills, capsules, liquid, vapors, or powders.

It is also understood and contemplated that aspects of this disclosure can provide more than two active ingredient components in the mixtures of compositions herein disclosed. Also, the disclosed methods can comprise the simultaneous or separate administration of multiple active ingredients. Thus, implementations may further include the administration of a third, fourth, etc. active ingredient, wherein the third, fourth, etc. active ingredient is administered separately, but at the same time as the other active ingredients, or hours or days after the first administration of active ingredients.

In some implementations, the C5 ketones, or pharmaceutical composition containing them, can be delivered in a controlled release system. Such methods may include the use of a pump for administration (e.g., use of an intravenous drip).

The compounds and compositions of the invention may also be utilized in pharmaceutically acceptable compositions in the methods provided herein. It would also be understood by a skilled artisan how to use the compositions described herein for therapeutic purposes without undue experimentation based on the teachings provided throughout the specification.

The amount of active ingredients or pharmaceutical compounds of the invention which will be effective in the treatment, inhibition and/or prevention of the diseases and/or conditions described herein can be determined by standard clinical techniques. Additionally, in vitro assays may be employed to help identify optimal dosage ranges. The precise dose to be utilized will also depend on the route of administration, and the seriousness of the disease or condition, and should also be decided according to the sound medical judgment of the clinician and each patient's individual circumstances. The specific therapeutically effective dosage level for any particular patient will depend upon a variety of factors including: the type and degree of the response to be achieved; the specific composition and other agent(s), if any, employed; the age, weight, general health, sex and diet of the patient; the time of administration, route of administration, and rate of excretion of the composition; the duration of the treatment; other drugs used in combination or coincidental with the composition; and any other factors well known in the medical arts. Effective dosages may also be extrapolated from dose-response curves derived from in vitro or animal model testing systems.

In some implementations, the mental performance level being increased is a measure of well-being and/or focus. In some implementations, the physical performance level being increased is energy.

In another aspect, the subject disclosure provides methods for increasing blood ketone levels in a subject, the methods comprising administering to the subject a therapeutically effective amount of a C5 ketone body, or pharmaceutically acceptable salt, ester, or chelate thereof In some implementations of this aspect, the C5 ketone body is 3-hydroxypentanoate.

In another aspect, the subject disclosure provides methods for treating and/or preventing one or more of obesity, diabetes, metabolic syndrome, Alzheimer's disease, Chronic Fatigue Syndrome (CFS), aging, fibromyalgia, dyslipidemia, hypercholesterolemia, dyslipidemia, Parkinson's disease, migraines, Traumatic Brain Injury (TBI), Attention Deficit Disorder (ADD)/Attention Deficit Hyperactivity Disorder (ADHD), Cancer, Cardiovascular Disease (CVD)/Coronary Artery Disease (CAD), Chronic Pain, neuralgia, depression, amyotrophic lateral sclerosis (ALS), epilepsy, Insufficient Cellular Energy (ICE) and mitochondrial dysfunction in a subject, the methods comprising administering to the subject a therapeutically effective amount of a C5 ketone body, or pharmaceutically acceptable salt, ester, or chelate thereof.

In yet another aspect, the subject disclosure provides methods for decreasing blood glucose levels and/or increasing BHB levels in a subject, the methods comprising administering to the subject a therapeutically effective amount of a C5 ketone body, or pharmaceutically acceptable salt, ester, or chelate thereof In some implementations of this aspect, the C5 ketone body is 3-ketopentanoate.

In yet another aspect, the subject disclosure provides methods for aiding in weight loss in a subject, the methods comprising administering to the subject a therapeutically effective amount of a C5 ketone body, or pharmaceutically acceptable salt, ester, or chelate thereof

In another aspect, the subject disclosure provides methods for lowering exertion during exercise in a subject, the methods comprising administering to the subject a therapeutically effective amount of a C5 ketone body, or pharmaceutically acceptable salt, ester, or chelate thereof.

In a further aspect, the subject disclosure provides compositions comprising a therapeutically effective amount of C5 ketone body, or pharmaceutically acceptable salt, ester, or chelate thereof.

### EXAMPLES

### Example 1

### BHP and BKP Effects on Subject Blood Glucose and Ketone Levels; and Subject Well-Being, Energy and Focus

Two (2) healthy, male subjects reported to the laboratory following a 12 hour overnight fast. Subjects were instructed to randomly consume either 10 grams of BHP or 10 grams BKP dissolved in 12 ounces of water. Subjects were then monitored for 150 minutes following drinking the mixture. Blood glucose, ketones, and visual analog scales (VAS) of well-being, energy, and focus were taken at 0, 30, 60, 90, 120, and 150 minutes. VAS scales consisted of a continuum of numbers from 1-10 with 1 being extremely low and 10 being extremely high.

Blood ketones were measured via finger prick using an Abbott Precision device which determines mMol of BHB. Blood glucose was measures using Abbott Precision glucose strips and was measured in mg/dL.

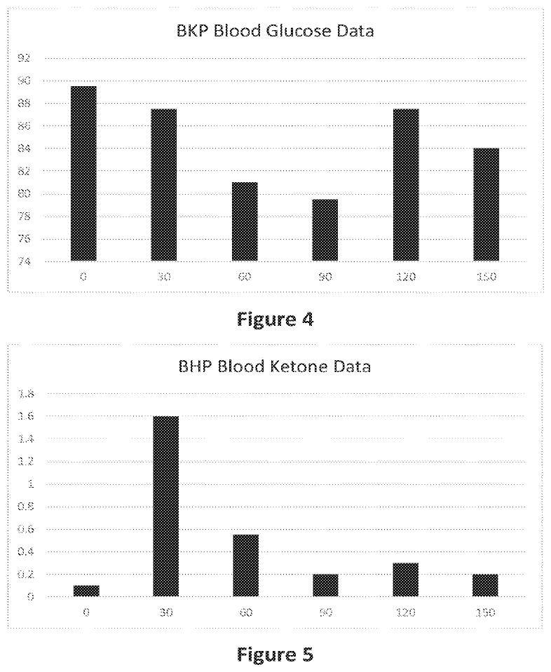

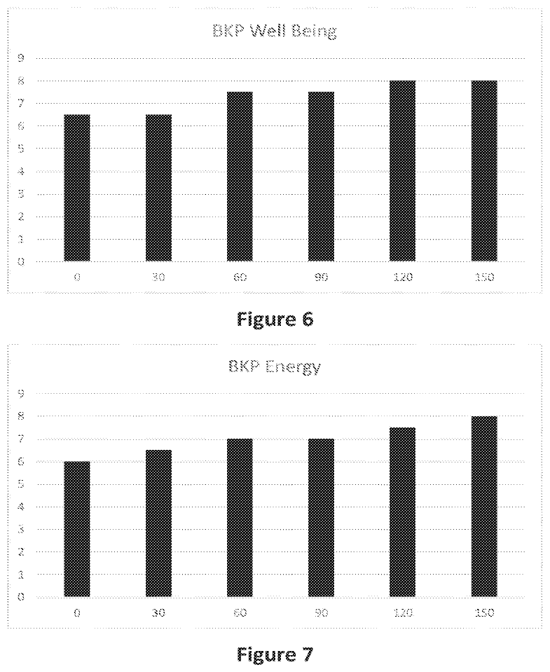

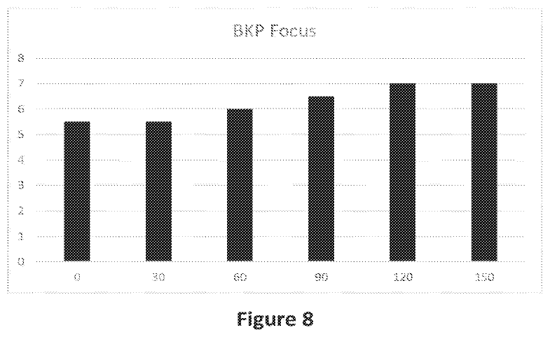

Results are summarized in Table 1 and FIG. 4 (FIG. 4 shows a graph of blood glucose level with BKP administration), Table 2 and FIG. 5 (FIG. 5 shows a graph of blood ketone level with BHP administration), Table 3 and FIG. 6 (FIG. 6 shows a graph of VAS of well-being with BKP administration), Table 4 and FIG. 7 (FIG. 7 shows a graph of VAS of energy with BKP administration), and Table 5 and FIG. 8 (FIG. 8 shows a graph of VAS of focus with BKP administration).

TABLE 1
Blood glucose levels (mg/dL) following BHP and BKP adminisixation.

| Time | | | | | | |
|---|---|---|---|---|---|---|
| points: | 0 min. | 30 min, | 60 min, | 90 min. | 120 min. | 150 min. |
| Subject 1 | | | | | | |
| BHP | 84 | 85 | 75 | 79 | 86 | 91 |
| BKP | 88 | 84 | 79 | 83 | 85 | 87 |
| Subject 2 | | | | | | |
| BHP | 84 | 76 | 66 | 79 | 81 | 90 |
| BKP | 91 | 91 | 83 | 76 | 90 | 81 |
| Combined | | | | | | |
| BHP | 84 | 80.5 | 70.5 | 79 | 83.5 | 90.5 |
| BKP | 89.5 | 87.5 | 81 | 79.5 | 87.5 | 84 |

TABLE 2
Blood ketone levels (mMol) following BHP and BKP administration.

| Time | | | | | | |
|---|---|---|---|---|---|---|
| points: | 0 min. | 30 min. | 60 min. | 90 min.. | 120 min. | 150 min. |
| Subject 1 | | | | | | |
| BHP | 0.1 | 1.8 | 0.7 | 0.2 | 0.2 | 0.2 |
| BKP | 0.1. | 0.1 | 0.2 | 0.1 | 0.1 | 0.1 |
| Subject 2 | | | | | | |
| BHP | 0.1 | 1.4 | 0.4 | 0.2 | 0.4 | 0.2 |
| BKP | 0.1 | 0.1 | 0.2 | 0.1 | 0.1 | 0.1 |
| Combined | | | | | | |
| BHP | 0.1 | 1.6 | 0.55 | 0.2 | 0.3 | 0.2 |
| BKP | 0.1 | 0.1 | 0.2 | 0.1 | 0.1 | 0.1 |

TABLE 3
VAS of Well-Being following BHP and .13K.P administration..

| Time | | | | | | |
|---|---|---|---|---|---|---|
| points: | 0 min. | 30 min. | 60 min. | 90 min. | 120 min. | 150 min. |
| Subject 1 | | | | | | |
| BHP | 7 | 5 | 5 | 7 | 5 | 6 |
| BKP | 7 | 7 | 9 | 9 | 9 | 9 |
| Subject | 2 | | | | | |
| BHP | 5 | 5 | 4 | 5 | 4 | 4 |
| BKP | 6 | 6 | 6 | 6 | 7 | 7 |
| Combined | | | | | | |
| BHP | 6 | 5 | 4.5 | 6 | 4.5 | 5 |
| BKP | 6.5 | 6.5 | 7.5 | 7.5 | 8 | 8 |

TABLE 4
VAS of Energy following BHP and BKP adininisintion.

| Time | | | | | | |
|---|---|---|---|---|---|---|
| points: | 0 min. | 30 min. | 60 min. | 90 min. | 120 min. | 150 min. |
| Subject 1 | | | | | | |
| BHP | 5 | 4 | 5 | 7 | 5 | 7 |
| BKP | 7 | 7 | 7 | 8 | 8 | 9 |
| Subject 2 | | | | | | |
| BHP | 3 | 3 | 2 | 3 | 4 | 5 |
| BKP | 5 | 6 | 7 | 6 | 7 | 7 |
| Combined | | | | | | |
| BHP | 4 | 3.5 | 3.5 | 5 | 4.5 | 6 |
| BKP | 6 | 6.5 | 7 | 7 | 7.5 | 8 |

TABLE 5
VAS of Focus foik-5wing BHP and BKP admstration.

| Time | | | | | | |
|---|---|---|---|---|---|---|
| points: | 0 min. | .30 min. | 60 min. | 90 min. | 120 min. | 150 min. |
| Subject 1 | | | | | | |
| BHP | 5 | 5 | 5 | 5 | 5 | 5 |
| BKP | 6 | 5 | 7 | 7 | 7 | 8 |
| Subject 2 | | | | | | |
| BHP | 2 | 3 | 3 | 3 | S | 5 |
| BKP | 5 | 6 | 5 | 6 | 7 | 6 |
| Combined | | | | | | |
| BHP | 3.5 | 4 | 4 | 4 | 5 | 5 |
| BKP | 5.5 | 5.5 | 6 | 6.5 | 7 | 7 |

### Example 2

### C5 Ketone Administration as a Method to Lower Exertion During Exercise

The purpose of this experiment was to see if C5 ketones could lower exertion during moderate and high intensity cycling exercise. Five (5) college aged males were given a placebo or BKP (10 grams) prior to and during 2 minute low, moderate and high intensity cycling bouts. BKP is able to lower perceived exertion at both moderate and high intensity exercise compared to a placebo. See Table 6, which shows the Borg Rating of Perceived Exertion (RPE), which is a way of measuring physical activity intensity level. Perceived exertion is how hard one feels like the body is working.

TABLE 6
Rating of Perceived Exertion (RPE)

| | | Low | Moderate | High | |
|---|---|---|---|---|---|
| | Baseline | Intensity | Intensity | Intensity | 30 min-Post |
| BKP | 7 ± 1 | 9 ± 1 | 13 ± 2 | 17 ± 2 | 8 ± 1 |
| Placebo | 7 ± 1 | 9 ± 1 | 14 ± 2 | 18 ± 1 | 9 ± 1 |

The results of these findings show that C5 ketone bodies lower blood glucose, increase blood ketone levels, and increase visual analog scales of well-being, focus, and energy in otherwise healthy males. The disclosure further shows that C5 ketones are able to lower perceived exertion at both moderate and high intensity exercise. This disclosure has significant implications ranging from therapeutic effects to cognitive and athletic performance in a wide array of populations.

### End of Examples

In some implementations, a kit may be provided with the C5 composition. The C5 composition may include one or more C5 ketone or pharmaceutically acceptable salt, ester, or chelate thereof, as described herein. The kits may further be used in the methods described herein. The kits may also include at least one reagent and/or instructions for their use. Also, the kit may include one or more containers filled with reagent(s) and/or one or more components of the disclosure. One or more container of the kits provided may also comprise a C5 ketone, preferably in a purified form. Also, one or more container of the kits provided may also comprise another carbon length ketone, preferably in a purified form. In some implementations, the various C5 ketones and other carbon length ketones may be provided in separate containers for mixing prior to use, or for individual ingestion/administration. The kits may also comprise a control composition, such as reagents that lack a ketone for use as a control reagent in experimentation. As it would be understood by those skilled in the art, detection or labeling methodologies can be used in the kits provided when utilized in an experimental or laboratory setting.

Several aspects of the disclosure are described below, with reference to examples for illustrative purposes only. It should be understood that numerous specific details, relationships, and methods are set forth to provide a full understanding of the disclosure. One having ordinary skill in the relevant art, however, will readily recognize that the aspects described can be practiced without one or more of the specific details or practiced with other methods, protocols, reagents, and animals. The aspects described are not limited by the illustrated ordering of acts or events, as some acts may occur in different orders and/or concurrently with other acts or events. Furthermore, not all illustrated acts, steps or events are required to implement a methodology in accordance with the aspects described. Many of the techniques and procedures described, or referenced herein, are well understood and commonly employed using conventional methodology by those skilled in the art.

Prior to setting forth aspects of the disclosure in detail, it may be helpful to the understanding thereof to define several terms, and these are accordingly set forth in the next section, below. Unless otherwise defined, all terms of art, notations and other scientific terms or terminology used herein are intended to have the meanings commonly understood by those of skill in the art to which this disclosure pertains. In some cases, terms with commonly understood meanings are defined herein for clarity and/or for ready reference, and the inclusion of such definitions herein should not necessarily be construed to represent a substantial difference over what is generally understood in the art. It will be further understood that terms, such as those defined in commonly used dictionaries, should be interpreted as having a meaning that is consistent with their meaning in the context of the relevant art and/or as otherwise defined herein.

The terminology used herein is for the purpose of describing particular implementations only and is not intended to be limiting. As used herein, the indefinite articles “a”, “an” and “the” should be understood to include plural reference unless the context clearly indicates otherwise.

The phrase “and/or,” as used herein, should be understood to mean “either or both” of the elements so conjoined, i.e., elements that are conjunctively present in some cases and disjunctively present in other cases.

As used herein, “or” should be understood to have the same meaning as defined above for “and/or”. For example, when separating a listing of items, “and/or” or “or” shall be interpreted as being inclusive, i.e., the inclusion of at least one, but also including more than one, of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of, ” “only one of,” or“exactly one of”

As used herein, the terms “including”, “includes”, “having”, “has”, “with”, or variants thereof, are intended to be inclusive similar to the term “comprising.”

As used herein, the term “subject” refers to an animal. Typically, the terms “subject” and “patient” may be used interchangeably herein in reference to a subject. As such, a “subject” includes an animal that is being treated for conditions described in the present disclosure, such as, but not limited to, obesity, diabetes, metabolic syndrome, Alzheimer's disease, Chronic Fatigue Syndrome (CFS), aging, fibromyalgia, dyslipidemia, hypercholesterolemia, dyslipidemia, Parkinson's disease, migraines, Traumatic Brain Injury (TBI), Attention Deficit Disorder (ADD)/Attention Deficit Hyperactivity Disorder (ADHD), Cancer, Cardiovascular Disease (CVD)/Coronary Artery Disease (CAD), Chronic Pain, neuralgia, depression, amyotrophic lateral sclerosis (ALS), and epilepsy, Insufficient Cellular Energy (ICE) and mitochondrial dysfunction; or the recipient of a mixture of components or compositions as described herein. The term “animal,” includes, but is not limited to, a mammal, such as a mouse, rat, dog, guinea pig, cow, horse, chicken, cat, rabbit, pig, monkey, chimpanzee, and human.

The term “effective amount” or “therapeutically effective amount” means that amount of active compound that elicits the biological or medicinal response in a tissue system, animal or human that is being sought by a researcher, veterinarian, medical doctor or other clinician, which includes alleviation of one or more of the symptoms of the disease or disorder being treated; reduction of the severity of one or more of the symptoms of the disease or disorder being treated; or otherwise provides the desired effect. The precise dosage will vary according to a variety of factors such as subject-dependent variables (e.g., age, medical history, etc.), the disorder or condition, and the treatment being affected. In particular aspects of the present disclosure directed to co-therapy or combination therapy, comprising administration of a therapeutically effective amount of a C5 ketone body, or pharmaceutically acceptable salt, ester, or chelate thereof, and other carbon length ketones, therapeutically effective amount means that amount of the combination of agents taken together so that the combined effect elicits the desired biological or medicinal response. For example, the therapeutically effective amount of co-therapy comprising administration of a C5 ketone body, or pharmaceutically acceptable salt, ester, or chelate thereof, and other carbon length ketones would be the amount of the C5 ketone body and the amount of the other carbon length ketone(s) that, when taken together or sequentially, have a combined effect that is therapeutically effective. Further, it will be recognized by one skilled in the art that in the case of co-therapy with a therapeutically effective amount, as in the example above, the amount of C5 ketone body, or pharmaceutically acceptable salt, ester, or chelate thereof, and other carbon length ketone, individually, may or may not be therapeutically effective.

The term “pharmaceutically acceptable,” as used herein, means approved by a regulatory agency of the Federal or a state government or listed in the U.S. Pharmacopeia or other generally recognized pharmacopeia for use in animals and/or in humans. “Pharmaceutically acceptable salt” refers to a salt of a compound of the disclosure that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2naphthalenesulfonic acid, 4-toluenesulfonic acid, camphorsulfonic acid, 4-methylbicyclo [2.2.2]-oct-2-ene-1-carboxylic acid, glucoheptonic acid, 3-phenylpropionic acid, trimethylacetic acid, tiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound is replaced.

The term “carrier” refers to a diluent, adjuvant, excipient, and/or vehicle with which the compositions are administered. Such pharmaceutical carriers can be sterile liquids, such as water and oils, including those of petroleum, animal, vegetable or synthetic origin, such as peanut oil, soybean oil, mineral oil, sesame oil and the like. Saline solutions and aqueous dextrose and glycerol solutions can also be employed as liquid carriers, particularly for injectable solutions. Suitable pharmaceutical excipients include starch, glucose, sucrose, gelatin, lactose, malt, rice, flour, chalk, silica gel, sodium stearate, glycerol monostearate, talc, sodium chloride, glycerol, propylene, glycol, water, ethanol and the like. The pharmaceutical composition(s) may also contain wetting or emulsifying agents or suspending/diluting agents, or pH buffering agents, or agents for modifying or maintaining the rate of release of the formulations. The compositions can take the form of solutions, suspensions, emulsion, tablets, pills, capsules, powders, gels, creams, sustained-release formulations and the like. Formulations can include standard carriers such as pharmaceutical grades of mannitol, lactose, sodium saccharine, starch, magnesium stearate, cellulose, magnesium carbonate, etc. Such compositions will contain an effective amount of the active ingredient(s) together with a suitable amount of carrier so as to provide the proper form to the subject based on the mode of administration to be used.

In some implementations, if for intravenous administration, the compositions are packaged in solutions of sterile isotonic aqueous buffer. Where necessary, the composition may also include a solubilizing agent. The components of the composition are supplied either separately or mixed together in unit dosage form, for example, as a dry lyophilized powder or concentrated solution in a hermetically sealed container such as an ampoule or sachet indicating the amount of active agents. If the composition is to be administered by infusion, it can be dispensed with an infusion bottle containing sterile pharmaceutical grade water or saline. Where the composition is administered by injection, an ampoule of sterile water or saline can be provided so that the ingredients may be mixed prior to injection.

In some implementations, if a packaging material is utilized to package the composition, it may be biologically inert or lack bioactivity, such as plastic polymers, silicone, etc. and may be processed internally by the subject without affecting the effectiveness of the components packaged and/or delivered therewith. Additionally, the compositions and components may be packaged with additional agents.

“Preventing” or “prevention” refers to a reduction in risk of acquiring disorder (i.e., causing at least one of the clinical symptoms of the disease/disorder not to develop in a subject that may be predisposed to the disorder but does not yet experience or display symptoms of the disorder). “Treating” or “treatment” of any disorder refers, in one implementation, to ameliorating the disorder (i.e., arresting or reducing the development of the disorder (e.g., Alzheimer's disease) or at least one of the clinical symptoms thereof). In another implementation “treating” or “treatment” refers to ameliorating at least one physical parameter, which may not be discernible by the subject. In yet another implementation, “treating” or “treatment” refers to modulating the disorder, either physically, (e.g., stabilization of a discernible symptom), physiologically, (e.g., stabilization of a physical parameter), or both. In yet another implementation, “treating” or “treatment” refers to delaying the onset of the disorder.

Without further elaboration, it is believed that one skilled in the art can, using the preceding description, utilize the present invention to its fullest extent. The following examples are offered by way of illustration, not by way of limitation. While specific examples have been provided, the above description is illustrative and not restrictive. Anyone or more of the features of the previously described implementations can be combined in any manner with one or more features of any other implementations in the present invention. Furthermore, many variations of the invention will become apparent to those skilled in the art upon review of the specification.

All publications and patent documents cited in this application are incorporated by reference in pertinent part for all purposes to the same extent as if each individual publication or patent document were so individually denoted. By citation of various references in this document, Applicants do not admit any particular reference is “prior art” to their invention.

In some implementations, one or more additives may be included in the R-beta-hydroxybutyrate salt blend composition, such as flavorings (e.g., natural and/or artificial), vitamins, minerals, binders, pharmaceutically acceptable carriers (e.g., liquid and/or other types of carriers) and/or any other appropriate additive. The additives may alter flavor, color, and/or texture. The additives may increase palatability and/or facilitate inclusion in a delivery vehicle (e.g., tablet, food product, beverage product such as a drink mix, etc.). The additive may be any appropriate solid and/or liquid to which the compound is added. For example, an additive may include liquid carriers, such as water, milk(s), bone broth, and/or any other appropriate drinkable liquid. In some implementations, the R-beta-hydroxybutyrate salt blend composition may include a pharmaceutically inert liquid carrier, such as water (e.g., tap water, filtered water, distilled water, etc.). The liquid carrier may include other drinkable liquids such as coconut water, watermelon water, electrolyte water, bone broth, and/or combinations thereof. The liquid carrier may include milks such as dairy milk, non-dairy milk, coconut milk, other milks, and/or combinations thereof. The liquid carrier may include an electrolyte solution, in some implementations. Use of a liquid carrier, such as bone broth may facilitate maintenance of ketosis or cause ketosis while providing satiety.

The described R-beta-hydroxybutyrate salt blend compositions may be administered via any appropriate administration method. For example, the described compositions may be administered enterally and/or parenterally. In some implementations, the described composition may be administered via a tablet and/or capsule. The described composition may be provided in a powdered form that allows the described composition to be sprinkled on food, mixed with a liquid to provide a beverage, and/or directly administered. The described composition may be provided in gel form. The compounds in the composition may be mixed, coupled to each other, and/or provided separately. For example, the composition may include beta-hydroxybutyrate coupled to another compound (e.g., beta-hydroxybutyrate ester and/or amino acid). In some implementations, the first mixture and one or more other compounds may be provided separately (e.g., in pills, packets, etc.). An individual may sequentially and/or concurrently be administered (e.g., swallow pills) the beta-hydroxybutyrate and other compounds.

The described R-beta-hydroxybutyrate salt blend compositions may be administered on an administration protocol to cause weight loss and/or maintain a weight of an individual; elevate and/or maintain blood ketone levels; increase and/or maintain ketosis; and/or improve glucose tolerance (e.g., fasting glucose levels may be reduced and/or glucose metabolism may be improved), in some implementations. For example, the described compositions may be administered once a day, via an extended-release preparation, and/or multiple times a day (e.g., 1 to 5 times a day, 2 to 5 times a day, 3 to 5 times a day, etc.). The described composition may replace other pharmaceuticals or dietary supplements taken to promote weight loss, maintain a weight, promote ketosis, elevate blood ketone levels and/or be utilized in combination with one or more other pharmaceuticals or dietary supplements, as appropriate. The described composition may replace other pharmaceuticals or dietary supplements taken for improving glucose tolerance, such as metaformin, and/or be utilized in combination with one or more other pharmaceuticals or dietary supplements, as appropriate, in some implementations.

In various implementations, the described R-beta-hydroxybutyrate salt blend composition(s) may include one or more of the described components, equivalent(s) of the described component(s), derivatives of the described component(s), complex(es) of the described component(s), salt(s) of the described component(s), and/or combinations thereof.

In various implementations, a pharmaceutically effective amount of one or more of the described R-beta-hydroxybutyrate salt blend composition(s) may be administered. Administration of the pharmaceutically effective amount may induce and/or maintaining ketosis; maintaining and/or promoting weight loss; increase mental processes (e.g., acuity including cognitive functioning, mood, energy, alertness, focus, performance, effects of aging, etc.); improve and/or maintain body composition; function as a therapeutic for one or more of the described conditions or disorders (e.g., treat neurological disorders); and/or combinations thereof. In some implementations, a pharmaceutically effective amount of one or more of the described R-beta-hydroxybutyrate salt blend composition(s) may be administered as a treatment for seizures and/or Alzheimer's or symptoms thereof.

Although various types of increases in mental acuity have been described, other features of mental acuity such as memory, focus, concentration, and/or understanding (e.g., speed of processing, accuracy of processing) may be increased by administration of an effective amount of the R-beta-hydroxybutyrate salt blend composition.

Although a subject and/or an individual have been described as a human, a subject and/or individual may be a person or a group of people.

In various implementations, R-beta-hydroxybutyrate salt blend composition may be administered simultaneously and/or sequentially with one or more other compounds (e.g., short chain, medium chain, and/or long chain fatty acids). For example, R-beta-hydroxybutyrate salt blend composition and one or more other compounds may be delivered mixed in a powdered, liquid, gel, and/or other appropriate form. In some implementations, the R-beta-hydroxybutyrate salt blend composition and one or more other compounds may be administered via pills, tablets, capsules, other oral administration forms, intravenously, nasal sprays, sublingual tabs/strips, or topical delivery, rectal, other appropriate administration forms, and/or combinations thereof.

Although the term beta-hydroxybutyrate is the terminology used in the described implementations, beta-hydroxybutyrate is also referred to as beta-hydroxybutyrate , (R)-3-Hydroxybutyric acid, (R)-3-Hydroxybutanoic acid, (3R)-3-hydroxybutanoic acid, (R)-3-Hydroxybutanoate, (R)-(−)-3-Hydroxybutyric acid, (R)-(−)-beta-Hydroxybutyric acid, 3-D-hydroxybutyrate, BHIB, BHB, 3-delta-hydroxybutyrate, delta-3-hydroxybutyrate, 3-D-hydroxybutyric acid, D-3-hydroxybutyric acid, 3R-hydroxy-butanoic acid, delta-beta-hydroxybutyrate, D-3-hydroxybutyrate, D-(−)-3-hydroxybutyrate, delta-3-hydroxybutyric acid, (−)-3-Hydroxybutyric acid, D-beta-hydroxybutyrate, (R)-(−)-b-Hydroxybutyrate, (R)-beta-Hydroxybutyric acid, delta-(−)-3-hydroxybutyrate, (R)-3-hydroxybutyrate, (R)-beta-Hydroxybutanoic acid, (R)-(−)-beta-hydroxybutyrate, (−)-3-Hydroxy-n-butyric acid, (R)-(−)-b-Hydroxybutyric acid, Butanoic acid, 3-hydroxy-, (R)-Butyric acid, 3-hydroxy-, D-(−)-(R)-3-82578-46-9, beta-D-Hydroxybutyric acid, D-beta-Hydroxybutyric acid, (3R)-3-delta-hydroxybutyric acid, 3-(R)-Hydroxybutyric acid, and/or (−)-beta-Hydroxybutyrate.

In various implementations C5 composition is described as included in a composition; administered in an amount, form, and/or schedule; and/or being in a particular form (e.g., complexed and/or coupled). C5 composition may be utilized in the various described implementations of C5 ketone bodies in the same or higher amounts as the described C5 ketone bodies, as appropriate.

It is to be understood the implementations are not limited to particular systems or processes described which may, of course, vary. It is also to be understood that the terminology used herein is for the purpose of describing particular implementations only, and is not intended to be limiting. As used in this specification, the singular forms “a”, “an” and “the” include plural referents unless the content clearly indicates otherwise. Thus, for example, reference to “a compound” includes a combination of two or more compounds and reference to “a C5 ketone body” includes different types and/or combinations of C5 ketone bodies. As another nonlimiting example, reference to “C5 ketone bodies” may include a single type of C5 ketone body. As another nonlimiting example, reference to “a composition” includes a combination of two or more compositions.

Although the present disclosure has been described in detail, it should be understood that various changes, substitutions, and alterations may be made herein without departing from the spirit and scope of the disclosure as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A method for increasing mental and/or physical performance levels in a subject, the method comprising administering to the subject a therapeutically effective amount of a C5-ketone body, or pharmaceutically acceptable salt, ester, or chelate thereof.

2. The method of claim 1, wherein the C5-ketone body is β-ketopentanoate.

3. The method of claim 1, wherein the mental performance level being increased is a measure of well-being.

4. The method of claim 1, wherein the physical performance level being increased is energy.

5. The method of claim 1, wherein the mental performance level being increased is focus.

6. The method of claim 1 further comprising administering to the subject at least one additional compound, and wherein at least one of the additional compounds comprises:
a short chain fatty acid;
an ester of short chain fatty acid;
a medium chain fatty acid;
an ester of medium chain fatty acid;
a long chain fatty acid; or
an ester of long chain fatty acid.

7. A method for increasing blood ketone levels in a subject, the method comprising administering to the subject a therapeutically effective amount of a C5-ketone body, or pharmaceutically acceptable salt, ester, or chelate thereof.

8. The method of claim 7, wherein the C5-ketone body is β-hydroxypentanoate.

9. The method of claim 7 further comprising administering to the subject at least one additional compound, and wherein at least one of the additional compounds comprises:
a short chain fatty acid;
an ester of short chain fatty acid;
a medium chain fatty acid;
an ester of medium chain fatty acid;
a long chain fatty acid; or
an ester of long chain fatty acid.

10. A method for decreasing blood glucose levels in a subject, the method comprising administering to the subject a therapeutically effective amount of a C5-ketone body, or pharmaceutically acceptable salt, ester, or chelate thereof.

11. The method of claim 10, wherein the C5-ketone body is β-ketopentanoate.

12. The method of claim 10 further comprising administering to the subject at least one additional compound, and wherein at least one of the additional compounds comprises:
a short chain fatty acid;
an ester of short chain fatty acid;
a medium chain fatty acid;
an ester of medium chain fatty acid;
a long chain fatty acid; or
an ester of long chain fatty acid.

13. A method for lowering exertion during exercise in a subject, the method comprising administering to the subject a therapeutically effective amount of a C5-ketone body, or pharmaceutically acceptable salt, ester, or chelate thereof.

14. The method of claim 13, wherein the C5-ketone body is β-ketopentanoate.

15. The method of claim 13, wherein the exercise is moderate intensity exercise.

16. The method of claim 13, wherein the exercise is high intensity exercise.

17. The method of claim 13 further comprising administering to the subject at least one additional compound, and wherein at least one of the additional compounds comprises:
a short chain fatty acid;
an ester of short chain fatty acid;
a medium chain fatty acid;
an ester of medium chain fatty acid;
a long chain fatty acid; or
an ester of long chain fatty acid.
